(19) United States
(12) Patent Application Publication
(10) Pub. No.: US 2024/0238352 A1
(43) Pub. Date: Jul. 18, 2024

(54) BACTERIAL CELL WALL BOUND TO A MUCOPOLYSACCHARIDE, USES THEREOF AND PROCESS FOR ITS PRODUCTION

(71) Applicant: AILEENS PHARMA S.R.L., Nova Milanese (IT)

(72) Inventors: Roberto Maria Antonio DI MARCO, Catania (IT); Sonia LONGO SORMANI, Nova Milanese (IT); Franca VERGALITO, Lucito (IT); Irene MAGNIFICO, Isernia (IT); Laura PIETRANGELO, Isernia (IT); Noemi VENDITTI, Bojano (IT); Marco Alfio CUTULI, Campobasso (IT); Giulio PETRONIO PETRONIO, Catania (IT)

(73) Assignee: AILEENS PHARMA S.R.L., Nova Milanese (IT)

(21) Appl. No.: 18/563,001

(22) PCT Filed: May 20, 2022
(86) PCT No.: PCT/EP2022/063824

§ 371 Date: Nov. 21, 2023

(30) Foreign Application Priority Data
May 21, 2021 (IT) 102021000013292

(51) Int. Cl. A61K 35/74; A61K 9/00; A61K 31/728; A61P 31/04; C12N 1/20; C12R 1/01

(52) U.S. Cl. CPC A61K 35/74; A61K 9/0014; A61K 31/728; A61P 31/04; C12N 1/205; C12R 2001/01

(57) ABSTRACT

The present invention relates to a bacterial cell wall or fragment thereof of a selected Gram-positive strain of Cutibacterium acnes linked with a mucopolysaccharide and to its uses in the prevention or treatment of an immunological disease especially of skin or mucosac. A process for linking the bacterial cell wall of the selected Cutibacterium acnes to a mucopolysaccharide is also herein disclosed.

### FIELD OF THE INVENTION

The present invention relates to a bacterial cell wall bound with a mucopolysaccharide, uses thereof and process for its production.

The present invention origins in the field of pharmaceutical, cosmetic and nutritional products.

Specifically, the present invention relates to the association of a bacterial cell wall of a selected strain of Cutibacterium acnes with a mucopolysaccharide provided with non-specific immunomodulating activity and to uses thereof in the pharmaceutical field.

Preferably, the herein disclosed bacterial cell wall associated with a mucopolysaccharide is for topical application and is suitable for treating dermatological disorders, infections or skin affections.

### BACKGROUND

Immunology essentially relates to the study of body's defense against infection. The immune system is typically divided into two categories: innate immune response and adaptive immune response. Any disruption/defects of the immune system can result in immunological skin diseases.

Excessive and undesirable immune responses can cause hypersensitivity or autoimmune diseases, while hypoimmunity can lead to infectious diseases and skin tumor

Undesirable immune responses is strictly correlated with inflammation, a biological response to a harmful stimulus or injury of a tissue of the organism.

Inflammation is considered as a protective response of the body aimed at eliminating the cause of tissue injury, clear out necrotic tissues damaged from the original insult and the inflammatory process, and initiate tissue repair.

This response of the organism involves either the immune cells and molecular mediators.

At present most of the more common inflammatory diseases of the skin are treated with systemic or topical administration of steroidal anti-inflammatory drugs.

However, despite the wide use and effectiveness of steroidal anti-inflammatory drugs in the treatment of dermatological diseases, some risks remain that the treated disease become resistant, and the symptoms may persist or return after the end of the treatment.

In addition, the abuse or misuse of steroidal formulations for topical administration aimed at treating inflammation of the skin may lead either to local or general side effects. For example, the topical treatment of skin surrounding the eyes with corticosteroids may lead to serious side effects to the eyes such as ocular hypertension and/or glaucoma.

In addition, in certain cases the treatment of immunological skin diseases with corticosteroids is not fully satisfactory.

Therefore, at present there is the need for having new products provided with anti-inflammatory and/or possibly even antimicrobial activity, which are alternative to the commercially available medicines.

In many cases when immunological skin diseases is accompanied with a bacterial infection, the therapeutical treatment includes a combination of a steroidal drug with an antibacterial drug.

However recent studies have shown that the antibiotic treatment of the skin kill most of the physiological skin microbiota, interfering with the complex ecosystem of the skin in which interactive and interdependent relationships exist between microbial constituents and between microbes and the host.

In addition, the abuse of antibiotic products for topical application in the treatment of skin infections increases the possibility of resistance to the local antibiotic therapy, forcing the physician to prescribe antibiotics of second generation.

Accordingly at present still exists a need to have new non-specific immunomodulating agents for treating immunological diseases of the skin.

There is also the need to have new products especially for topical application, provided with anti-inflammatory activity whose use even for a prolonged time is not affected by serious side effects.

There is also a need to have new products for treating immunological skin diseases especially those having an inflammatory component, whose topical application does not impair the immune response.

One of the aims of the present invention resides in providing a product provided with immunomodulating activity whose topical use substantially does not impair the skin microbiota.

It is also desirable to provide a product having local anti-inflammatory activity which is active also in reducing the bacterial load of pathogenic microorganisms. On the other hand, by contrasting the pathogens, a more rapid restoration of homeostasis conditions is achieved through the normalization of the skin resident floras/microbiota.

Another aim of the invention resides in the provision of a non-steroidal product provided with anti-inflammatory activity, which is specifically aimed at the topical application on the skin or mucosae such as vaginal mucosae.

### SUMMARY OF THE INVENTION

The present invention origins from the finding that a bacterial cell wall or fragment thereof of a selected Gram-positive bacterial strain belonging to genus Cutibacterium, species acnes, when linked to specific mucopolysaccharides, exerts an immunomodulating activity and promote the growth and migration of fibroblasts. In addition, the inventors have observed that the association of the selected strain of Cutibacterum acnes with mucopolysaccharides as described herein provides an inhibitory effect on pathogen microorganisms of the skin and regulates the biological mechanism of the local immune response blocking or slowing the inflammatory cascade.

These properties make the selected Gram-positive strain of Cutibacterum acnes or a cell wall fragment thereof linked with a mucopolysaccharide as described herein a suitable candidate for medical applications preferably in the dermatological and/or gynaecological field, for example in the treatment of immune-related skin diseases and in skin infections, especially in presence of inflammation.

In a first aspect, the present invention provides a bacterial cell wall or fraction or lysate thereof associated or combined with a mucolysaccharide wherein the strain is Cutibacterium acnes deposited under the Budapest Treaty with accession number (filing number) DSM 28251 at the International Deposit Authority Leibniz-Institut DSMZ—Deutsche Sammlung von Mikroorganismen und Zellkulturen GmbH.

Typically, a complex or conjugate is formed by the association of the strain cell wall or fraction or lysate thereof of Cutibacterium acnes DSM 28251 with a mucopolysaccharide.

In an aspect the invention provides medical uses of the above-mentioned bacterial cell wall of strain Cutibacterium acnes deposited with accession number DSM 28251. In accordance with this aspect, the strain of the invention is for topical or systemic administration, especially for topical use.

Systemic administration means a route of administration of a medicament, nutrition including the strain of the invention into the circulatory system so that the entire body is affected. Administration can take place via enteral, oral administration or parenteral administration for example by injection, infusion, or implantation.

Topical administration or application is the preferred route of administration of the bacterial cell wall or fraction thereof of Cutibacterium acnes DSM 28251 associated/linked to the mucopolysaccharide.

The bacterial cell wall of Cutibacterium acnes DSM 28251 linked with a mucopolysaccharide and compositions containing the same may be applied on the skin in any forms suitable for topical application.

The Cutibacterium acnes strain DSM 28251 from which originates the herein disclosed bacterial cell wall, may be derived by spontaneous mutation, induced mutation, conjugation and selection, hybridization and selection or other methods of genetic manipulation, and can be tracked back to it.

The bacterial strain from which originates the bacterial cell wall may be isolated and selected from healthy skin, among the multitude of strains that composes the skin microbiota.

According to another aspect, the invention relates to a bacterial cell wall or fragment/lysate of Cutibacterium acnes deposited with accession number DSM 28251 linked with a mucopolysaccharide, for use in the prevention or treatment of an immunological skin or mucosae disease.

Preferably, the bacterial cell wall associated with a mucopolysaccharide of the invention finds applications in the dermatological and gynaecological field, for example in the treatment of skin or mucosal inflammation or infections from bacteria, fungi or protozoa.

For example, the herein disclosed association is effective in the prevention and or treatment of bacterial infections which are the cause of some type I hypersensitivity reactions for example urticaria, and type III hypersensitivity reactions, for example allergic cutaneous vasculitis. The herein disclosed association is suitable also in the treatment of abnormal epidermal barrier function and dysregulated immunity causing atopic dermatitis an allergic based skin disease with increased susceptibility to skin infections and cutaneous colonization by pathogenic microorganisms such as Staphylococcus aureus.

In an aspect it is provided the bacterial cell wall or fraction/lysate of Cutibacterium acnes DSM 28251 linked with a mucopolysaccharide for use in the treatment of a skin disease caused by superantigens, virulence factors produced by bacteria such as S. aureus which could induce corticosteroid resistance.

In addition, the association of the invention is effective in the treatment of infections from fungi especially yeasts of the genus Candida spp, especially Candida albicans, or dermatophytes such as Malassezia spp, both are among the most common causative agents of opportunistic infections of the human body. In addition, the association or complex of cell wall fragment of C. acnes strain DSM 28251 and mucopolysaccharide of the invention is used in the treatment of fungal infections which are resistant to the antifungal products available on the market.

The applicant observed that the cell wall fragment of C. acnes strain DSM 28251 linked with a mucopolysaccharide as herein disclosed exerts an immuno-modulatory activity and activates the local immune system which is the first line of defense of the human organisms against antigens.

It has been also observed that the herein disclosed complex is provided with a cicatrizing action on skin.

The cosmetic use of the above-identified bacterial strain for ameliorating an aesthetic aspect of the skin such as redness or coupe rose is also herein disclosed. Accordingly, in an aspect the invention concerns with a fragment or lysate of the bacterial wall of Cutibacterium acnes DSM 28251 bound/associated with a mucopolysaccharide or a pharmaceutical composition containing said association and a pharmaceutically acceptable carrier, for use in the treatment of wounds, abrasion, ulcerations of the skin for example pressure ulcers and for repairing damages tissues of the body, especially skin.

The reactive functional groups in mucopolysaccharides such as the carboxyl and hydroxyl groups in their structure makes them suitable for binging with the bacterial wall of Cutibacterium acnes DSM 28251.

The fragment or lysate of the bacterial cell wall of the strain Cutibacterium acnes DSM 28251 may be obtained by delipidation of the bacterial cell wall and subsequent crushing of the strain.

In a further aspect the present invention concerns with a process for the production of a bacterial cell wall of Cutibacterium acnes DSM 28251 linked/associated with a mucopolysaccharide, comprising the steps of

a) oxidation or acetylation of the primary alcohol with one of: periodate, acyl halides, glutaraldehyde, iodacetic acid, chloroacetic acid. Preferably, the oxidation takes places on the hydroxyl of carbon six of the N-acetylglucosamine moiety of the mucopolysaccharide such as hyaluronic acid due to the improved accessibility of the reagents to the primary alcohols. This step may be carried out according to the disclosure of Kristiansen, K. A., Potthast, A., & Christensen, B. E., “Periodate oxidation of polysaccharides for modification of chemical and physical properties.” Carbohydrate Research, 345(10), 1264-1271. (2010).

b) reaction, in an aqueous or organic medium, between the activated mucopolysaccharide comprising aldehyde and carboxyl groups with amine groups present in the bacterial wall forming the Schiff base or amide groups.

For example, the reaction of step b) may be carried out using water as solvent and a buffer to adjust the pH in the range of 8.5-9, in the alternative the solvent advantageously include ethanol and tetrahydrofuran (THF) and a phosphate buffer preferably to adjust the pH at 7.4+/−0.4.

The mucopolysaccharides can be chemically modified in two different ways: by conjugation or by cross-linking.

Both methods are based on the same chemical reaction, in conjugation the compound is bound onto a mucopolysaccharide chain such as hyaluronic acid chain by a single bond.

In the case of cross-linking, the mucopolysaccharide chains are bound together by two or more bonds.

The conjugation reaction can occur either using the reactivity of the groups present in the biopolymers or by creating suitable sites for chemical conjugation on the polymer to obtain an activated mucopolysaccharide.

Typically, the conjugation groups of the bacterial wall fragment are preferably amine, carboxyl and thiol groups.

The

### BRIEF DESCRIPTION OF THE FIGURES

The invention will now be described in detail and in reference to the attached Figures wherein:

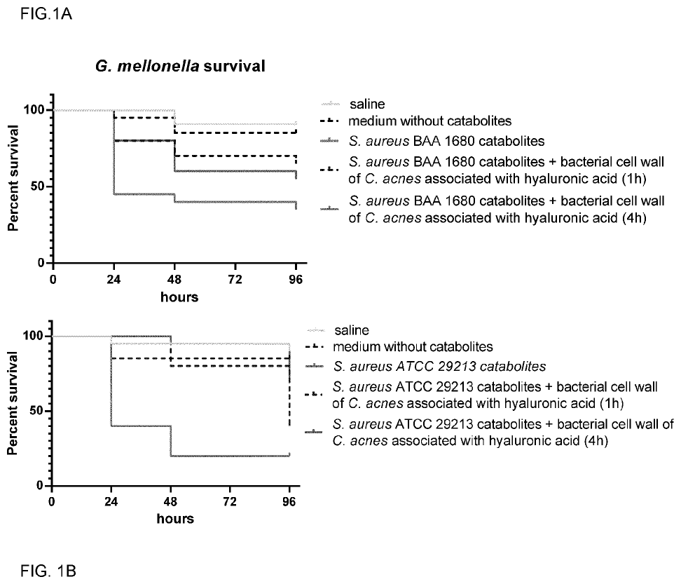

The FIGS. 1A and 1B show two graphs illustrating the survival rate of Galleria mellonella larvae inoculated with supernatants from S. aureus BAA1680 and S. aureus ATCC 29213 cultures according to Example 1.

### DETAILED DESCRIPTION OF THE INVENTION

The invention origins from the finding that certain activities of Cutibacterium acnes strain DSM 28251 are unexpectedly improved when the bacterial cell wall of this strain of fragment thereof is associated or linked to a mucopolysaccharide, advantageously to give a complex of mucopolysaccharide with C. acnes strain DSM28251.

In certain embodiments, the mucopolysaccharide or salt thereof is linked or complexed with a bacterial wall derived glycoprotein and peptide glycan complex of Cutibacterium acnes strain DSM 28251.

According to a main aspect, the invention concerns with a bacterial cell wall or fraction or lysate thereof linked to a mucopolysaccharide as defined in claim 1.

In particular, the bacterial cell wall or fragment thereof linked to a mucopolysaccharide form a complex or conjugate.

Therefore, according to certain aspects, a complex is provided comprising the bacterial cell wall or fragment or lysate of C. acnes DSM 28251 linked to a mucopolysaccharide.

Specifically, the bacterial cell wall of Cutibacterium acnes strain DSM 28251 or a fragment/fraction thereof when linked or associated to a mucopolysaccharide is provided with an immunomodulating action which makes it effective in the treatment of immunological skin diseases.

Advantageously, the cell wall of Cutibacterium acnes strain DSM 28251 or a fragment/fraction thereof when linked to a mucopolysaccharide activates an immune response. The immune response of the treated human body may be systemic or local depending if the complex of the fragment of the Cutibacterium acnes strain DSM 28251 linked to the mucopolysaccharide is orally administered or applied on the skin or mucosa of the human body.

The above activities are proven by the experimental tests carried out by the inventors and reported in the following example. These tests provide a scientific basis for the use of the above-identified association as an immunomodulatory agent, especially for topical applications.

Suitable mucopolysaccharides linked or associated with the bacterial cell wall of the described strain are physiologically acceptable mucopolysaccharides or salts thereof belonging to the following groups based on core disaccharide structures:

group 1: heparin/heparan sulfate (HSGAGs); group 2: chondroitin sulfate/dermatan sulfate (CSGAGs), group 3: keratan sulfate, chitosan; group 4: hyaluronic acid or physiologically acceptable salts thereof.

In certain preferred embodiments the mucopolysaccharides are selected from hyaluronic acid (HA) and salts thereof, chondroitin-4-sulfate (C4SA), chondroitin-6-sulfate (C6SC), chitosan, dermatan sulfate (DS-condroitin-solfate B), heparin sulfate (HS), heparin (HP) and keratan-sulfate (KS) and physiologically acceptable salts of the above mucopolysaccharides.

Amongst physiologically acceptable mucopolysaccharides, hyaluronic acid or a salt thereof are preferred. Suitable salts of hyaluronic acid include the salts of sodium, potassium, calcium.

In certain embodiments the molecular weight of HA is from 3×10⁴ and 8×10⁶ MW (T. C. Laurent et al., Fractionation of hyaluronic acid. The polydispersity of hyaluronic acid from the bovine vitreous body, Biochim. Biophys Acta, 1960, 42, 476).

Typically, the molecular weight of the mucopolysaccharides, such as hyaluronic acid, is average molecular weight (MW) which may be determined by conventional techniques such as SEC-MALLS or multi angle laser light diffusion—chromatography at dimensional exclusion, or with a method as described in Ueno et al., 1988, Chem Pharm Bull. 36, 4971-4975; Wyatt 1993, Anal Chim Acta 272: 1-40; Watt Technologies 1999 “Light scattering University Dawn Course Manual and “Dawn Eos Manual” Wyatt Technology Corp. Santa Barbara CA (USA).

In certain aspects of the invention, a composition, especially a pharmaceutical or nutritional composition, containing the association as defined in claim 1, is hereby provided.

In accordance an aspect, the invention concerns with the herein disclosed bacterial cell wall or fragment thereof linked to a mucopolysaccharide for use as a medicament.

In particular, the bacterial cell wall or fragment thereof linked to a mucopolysaccharide according to anyone of the described embodiments is for use in the treatment of immunological skin diseases.

Immunological skin diseases which may be treated according to the invention include infectious, allergic, autoimmune and other types based on the aetiology, pathogenesis and immune responses.

The features of the bacterial cell wall of claim 1 provides either immunomodulation and anti-inflammatory actions enabling the physician to treat a wide range of disease especially allergic, autoimmune, infectious disease of the skin or mucosa of a mammal such as human beings.

In addition, the bacterial component of the bacterial cell wall of associated with the mucopolysaccharide when orally administered or applied on the skin of an individual, triggers an immune response against pathogenic microorganisms. This effect/activity makes the association useful in the treatment of infections, especially skin and mucosal infections.

In particular, the herein described fragment of bacterial wall of C. acnes strain DSM 28251 is effective in the treatment of most of common bacteria, mainly gram-positive bacteria, especially cocci bacteria such as Staphylococcus aureus or Escherichia coli and pathogenic yeasts such as those of the genus Candida, for example Candida albicans.

In accordance with another aspect, a complex is provided comprising a fragment of bacterial wall of the Cutibacterium acnes deposited with accession number DSM 28251 complexed with a mucopolysaccharide as herein disclosed.

Advantageously, in the mucopolysaccharide moiety is complexed or linked with peptidoglycans of the bacterial wall fragment and possibly is complexed or linked also with lipoteichoic acid and/or teichoic acid.

The DSM 28251 strain from which is obtained the herein disclosed bacterial cell wall is genotypically characterized and is identifiable in a clear and defined manner by specific traits identified within the genome. This strain has evolved spontaneously, without any direct intervention or genetic manipulation, and has the relevant characteristics for industrial application.

In order to verify and ascertain the characteristics of the DSM 28251 strain, and to rule out the possible overlap of this strain with the strains described in the prior art, a genotype characterization was made by DSMZ.

In certain aspects the invention also concerns with a bacterial cell wall of Cutibacterium acnes DSM 28251/linked with a mucopolysaccharide and compositions containing the same for medical uses in the treatment of:
a gynecological disease such as vaginitis, vaginal infection or inflammation or
a proctological disorder for example hemorrhoids, anal rhagades or skin scars or the perianal area or
wounds, injuries, abrasion, ulcerations of the skin, for example pressure ulcers or to heal wounds.

### Chemical Characterization of Parietal Fragment of C. Acnes DSM 28251

The cell wall of C. acnes DSM 28251 and fragment thereof has a specific cell wall, containing phosphatidylinositol, triacylglycerol, and lipids.

In particular, the cell wall or fragment thereof of C. acnes DSM 28251 contains peptidoglycan (PNG) of a type different from that of other Gram-positive bacteria, in that advantageously, the peptide chain may contain the L-acid L-diaminopelic acid and D-alanine.

Advantageously, the C. acnes DSM 28251 lipoglycans have a lipid anchor based on fatty acids and a polysaccharide moiety containing mannose, glucose, and galactose, together with an amino sugar, especially a diaminohexuronic acid.

The total and free hexosamine content may be determined as described in Elson and Morgan, 1933 (Elson, L. A. and Morgan, W. T. J. A colorimetric method for the determination of glucosamine and chondrosamine. Biochemical Journal (1933) 27:1824-1828), using glucosamine hydrochloride as the standard. The content of free hexosamine is extremely low (value expressed as g/100 g dry weight): 0.121±0.011 (range 0.113-0.131). The total content of hexosamines is 9.21±2.02 (range 7.974-11.535).

Analysis of total amino acid content of the fragment of C. acnes DSM 28251:

a sample of a fragment of C. acnes DSM 28251 was subjected to total amino acid analysis by chromatographic analysis.

| | Mean value ± SD |
|---|---|
| Aminoacid | g/100 g sample X (1) |
| Asp | 3.75 ± 0.18 |
| Thr | 2.84 ± 0.13 |
| Ser | 2.88 ± 0.08 |
| Glu | 7.85 ± 0.15 |
| Gly | 4.18 ± 0.05 |
| Ala | 4.50 ± 0.29 |
| Val | 3.16 ± 0.09 |
| Cys | 0.42 ± 0.02 |
| Met | 1.08 ± 0.03 |
| lle | 2.48 ± 0.09 |
| Leu | 3.49 ± 0.64 |
| Tyr | 1.40 ± 0.06 |
| Phe | 2.46 ± 0.14 |
| Lys | 4.60 ± 0.14 |
| Hist | 1.75 ± 0.06 |
| Arg | 3.34 ± 0.09 |
| Pro | 1.60 ± 0.05 |
| Total | 51.79 ± 1.67 |

In addition, the fragment of cell wall of C. acnes DSM 28251 linked to a mucopolysaccharide provides a unique activity against Staphylococcus aureus, Escherichia coli and Candida, for example Candida albicans.

The above data evidence that the fragment of cell wall of C. acnes DSM 28251 linked to a mucopolysaccharide has chemical composition and biological activity which is unique and therefore different from fragment of cell wall of other bacteria gram + or gram −. This is confirmed by the scientific literature, for example by Cummins, C. S., & Hall, P. (1986). Acetate and pyruvate in cell wall polysaccharides of Propionibacterium acnes, P. avidum, and P. granulosum. Current Microbiology, 14(2), 61-63; McBride, W. H., Dawes, J. O. A. N., Dunbar, N. O. R. E. E. N., Ghaffar, A., & Woodruff, M. F. (1975).

### Process for Obtaining the Bacterial Cell Wall Fraction/Fragment or Lysate

A suitable bacterial cell wall, fragment or lysate of the strain Cutibacterium acnes DSM 28251 which is linked to the mucopolysaccharide, may be obtained by conventional or general methods of cell disruption.

A suitable destruction/disruption/crushing of the cell wall of the strain may be achieved either by subjecting the above strain to mechanical methods/lysis treatments or non-mechanical methods/lysis treatments.

In certain embodiments, before bacterial cell wall disruption or crushing, the starting strain may be inactivated by using conventional methods, for example by heating and/or treating the strain with formaldehyde.

In accordance with certain embodiments, the cell wall fragment of Cutibacterium acnes DSM 28251 is obtained by crushing the cell wall, by a mechanical method in particular by solid shear or fluid shear methods

### Bacterial Cell Wall Disruption with Mechanical Methods/Apparatus

Suitable mechanical methods for disrupting, crushing the bacterial cell wall of the present strain and obtaining suitable wall fragments include either solid shear or fluid shear methods.

Solid shear includes the use of bead mill, X-press or Hughes press.

Liquid/fluid shear includes sonication, high-pressure methods, for example Hughes press or the French press and/or the homogenization with a homogenizer or the use of a microfluider homogenizer.

The technique with bead mill (or abrasion) typically includes the agitation of a suspension of the strain with glass beads.

Typically, the disruption of the bacterial cell wall with a bead mill method is carried out in a bead mill which includes a jacketed grinding chamber with a rotating shaft running through its centre. The shaft is fitted with agitator(s) imparting the kinetic energy to beads in the chamber, forcing them to collide with each other (Chisti & Moo-Young, 1986; Middelberg, 1995). Suitable beads may be 0.10-0.15 mm in diameter for effective disruption of the bacteria. Large industrial apparatuses may use beads of 0.4-0.6 mm in diameter because of the mechanism for separating the beads from the suspension (Kula & Shutte, 1987). Suitable tip speed is of at least 10 m⁻¹ for the disruption of the bacteria (Kula & Shutte, 1987). The cell concentrations may vary from 40-50% wet weight in the broth introduced into the chamber.

Suitable shears also comprise sonication and high-pressure methods including the Hughes press or the French press, in which a frozen suspension of cells is forced through a small opening by high pressures (Engler, 1985).

Sonication includes the use of ultrasound, sound waves typically with frequency higher than 15-20 kHz which can disruption cell wall in suspension. Suitable acoustic power for example, when sonicating 5-30 ml of a 20% bacterial suspension in a conventional liquid medium using 35-95 W of acoustic power. In certain embodiment, the fragment of cell wall as herein described, is obtained by crushing the C. acnes DSM 28251 strain by a mechanical treatment, for example by sonication.

Alternatively, mechanical disruption may be obtained in a high-pressure valve homogenizer by passing a cell suspension of the strain under high pressure through an adjustable, restricted orifice discharge valve as reported by Engler, 1985. Typically, a basic homogenizer design comprises a positive-displacement pump that forces a cell suspension through the centre of a valve seat and across the seat face. Adjusting the force on the valve controls the pressure. The fluid flows radially across the valve and strikes an impact ring (Middelberg, 1995). Disruption results from non-specific tearing apart of the cell wall.

Exemplary homogenizer type is the Manton-Gaulin APV design (Middelberg, 1995). For example, the temperature is raised of about 21 C per 10 MPa in a homogenizer. There is a strong influence of the operating pressure on the disruption process in the homogenizer. By operating the homogenizer at higher pressures, it is possible to decrease number of passes of the cell slurry through the homogenizer for a given degree of disruption (Chisti & Moo-Young, 1986; Bury et al., 2001).

A microfluider homogenizer may also be used as equipment for obtaining fragmented cell wall. In this apparatus two streams of a cell suspension are impacted at high velocity against a stationary surface and the energy input is dissipated almost instantaneously at the point of impact leading to disruption of cells (Middelberg, 1995; Agerkvist & Enfors, 1990). The residence time of the strain suspension in the Microfluidizers disruption chamber, which is the hottest part of the device, is 25-40 ms. Cooling in place may be achieved by immersion of the disruption chamber in an ice bath (Sauer et al., 1989; Geciova, personal experience). The fraction of disrupted cells increases with increasing pressure and number of passes.

### Bacterial Cell Wall Disruption with Non-Mechanical Methods

A non-mechanical disruption method is based on decompression obtained by introducing a pressurized subcritical or supercritical gas into the cells causing disruption after release of applied pressure by expansion.

Another non mechanical disruption of the cell wall may be obtained by osmotic shock wherein a cell-strain suspension is diluted in a liquid medium/broth after equilibration in high osmotic pressure under conventional conditions.

An alternative method for cellular lysis is thermolysis which involves heat treatment of the cells for example under conventional conditions. Another non mechanical cell lysis may be obtained by chemical permeabilization especially with a substance selected from Antibiotics such as beta lactam antibiotic for example penicillin, chelating agents for example EDTA, chaotropes for example urea, guanidine, ethanol, detergents for example Triton X series, sodium dodecyl sulphate, sodium lauryl sarcosinate, solvents such as toluene, acetone, chloroform, hydroxides such as sodium hydroxide, hypochlorites such as sodium hypochlorite and mixtures thereof.

Cellular lysis of the strain may also be obtained by enzymatic lysis for example by using a protease and glucanase to attack, at first, the mannoprotein complex of the cell wall and then the glucan backbone (Kitamura, 1982). A suitable product for the strain wall lysis is commercial product Zymolase-20T (Seikagaku America, Inc., Rockville, MD). Lysozyme may be also used for lysis of peptidoglycan layers as it catalyses hydrolysis of b-1,4-glycosidic bonds.

In accordance with a preferred embodiment a fragment of wall of C. acnes DSM 28251 strain may be obtained by treating the bacterial strain with ammonium sulphate, preferably at a temperature lower than room temperature for example in the range of 10 to 2° C., and advantageously after the treatment the suspension is centrifuged and the precipitated fragment is collected.

Advantageously, before the treatment with ammonium sulphate, the C. acnes DSM 28251 strain is dried and centrifuged optionally with water. Optionally, after centrifugation the supernatant resulting from centrifugation is heated for example at a temperature of 40 to 95° C., preferably at 75-85° C. and then cooled for example with cool water, preferably at 3 to 15° C. Thereafter, the precipitation step is performed incubating with a solution of ammonium sulphate at a concentration from 20 to 60% v/v for example at 2 to 10° C. Advantageously, after incubation the obtained suspension is centrifuged and the precipitated fragment may be collected. For example, the bacterial pellet is delipidated by Soxhlet treatment using organic solvents selected from Ether-Ethanol, Chloroform, Methanol-Chloroform and a mixture thereof, then it is dried for example under the hood laminar flow. After drying, the pellet is homogenized by 2 steps of Ultraturrax treatment, preferably 1 minute each, adding distilled water, preferably in the proportion of 1:2 p/V. After centrifugation, the supernatant is warmed at 80° C. and then cooled under cool water, preferably at 3 to 15° C., and finally on ice. Subsequently, the fragment precipitation step is performed by incubating with 40% v/v cool ammonium sulphate for 24 hours at 4° C. After incubation, the suspension is centrifuged, and the precipitated fragment was collected and lyophilized.

### Delipidation

In some embodiments, the fragment of the cellular wall of Cutibacterium acnes deposited with accession number DSM 28251 is delipidated, i.e. treated so as either to remove or considerably reduce the lipid component of the cellular wall of the bacterium by means of chemical/biotech techniques.

Advantageously, the delipidation step is carried out before the bacterial cell wall disruption.

For example, the Cutibacterium acnes deposited with accession number DSM 28251 is delipidated prior to crushing to produce the cellular wall fragments. Typically, the delipidated fragments of cellular wall of the strain of the invention comprise sugars and peptide chains, typically bound to one another into glycopeptides which form a close knit mesh. Typical sugars of the cellular wall comprise N-Acetylmuramic acid and N-Acetylglucosamine.

For example, a process for obtaining a cell wall fragment of the C. acnes strain DSM 28251 includes the step of providing a strain of Cutibacterium acnes DSM 28251 included in a broth or culture medium, washing and delipidating the step by a soxhlet extractor.

The delipidated bacteria are then suspended in water and then subjected to mechanical lysis for example with a mechanical stirrer such as a homogenizer Ultratturrax and the content is treated with ammonium sulphate to precipitate the fragments of the bacterial wall.

The fragments can then be cleaned for example by washing with water to obtain the desired bacterial cell wall fragments.

### Bacterial Cell Wall Association/Linkage with Mucopolysaccharide

The association or link of the bacterial cell wall with the mucopolysaccharide may be obtained by reacting a fragment of cell wall of C. acnes strain DSM 28251 with a solution of a mucopolysaccharide or salt thereof in a suitable solvent.

In certain embodiments, the mucopolysaccharide or salt thereof is linked or complexed with a bacterial wall derived glycoprotein and peptide glycan complex (EDS).

An embodiment of the method for associating/linking the bacterial cell wall of strain DSM 28251 with the mucopolysaccharide comprises the following steps:

a salt of a suitable mucopolysaccharide for example, sodium, potassium or calcium hyaluronic acid (HA) salt, is dissolved in water with a buffer comprising sodium acetate to reach a pH in the range of 5 to 6, preferably of 5.3 to 5.7, for example pH 5.5. Thereafter, iodoacetate or chloroacetate is added for example in an amount of 1 to 10% by weight, preferably of 3 to 6%, for example 4% by weight with respect to the amount of starting hyaluronic acid salt, preferably stirring and then adding an alkalinizing agent preferably calcium carbonate to reach a basic pH preferably in the range of 8 to 10, preferably 8.5 to 9.5, more preferably 8.8-9.

To this solution is added a fragment of wall of C. acnes DSM 28251 according to an embodiment described herein to associate/link HA with wall of C. acnes DSM 28251. Preferably, the obtained bacterial cell wall bound/associated to mucopolysaccharide is leaved in the solution overnight. In certain embodiments, at least one branched aminoacid preferably selected from leucine, valine, iso-leucine or arginine, or glycerol or glycine is added to the solution of the cell wall associated to HA to improve the linkage between HA and the wall of C. acnes DSM 28251.

As an example, a conjugate of a fragment of cellular wall of DSM 28251 strain and HA may be prepared and obtained as follows: 5 g of sodium hyaluronate are dissolved in 100 ml of acetate buffer 0.05 M, pH 5.5 with agitation to obtain a solution which is not excessively viscose 856 mg of sodium monoiodoacetate or mono chloroacetate are then added to the solution and oxidation reaction is allowed at ambient temperature away from light for 30 minutes. The reaction is then blocked by adding 1 ml of glycerol 5 M. After 15 minutes of reaction, the pH is adjusted to about 9 by adding sodium carbonate in powder form, after which 5 g of fragment of wall of C. acnes strain DSM 28251 are added to the solution (100 stimulating units) and association/linkage is left to occur at ambient temperature for several hours with agitation and overnight at 4° C. without agitation. The aldehydic groups which are still free may be blocked by adding 5 ml of a solution of 1M of an amino acid, such as arginine, leucine, valine or iso-leucine. The solution is dialyzed in water and lyophilized after 30 minutes.

### Definitions in the Present Invention

“Strain DSM 28251” is meant to include a bacterial strain of the genus Cutibacterium, species acnes, filed on 18 Dec. 2013 (identification reference ULTIMO) and converted to a deposit under the Budapest Treaty on 22 Dec. 2019 with accession number DSM 28251 at the International Deposit Authority Leibniz-Institut DSMZ—Deutsche Sammlung von Mikroorganismen und Zellkulturen GmbH; Typically, Cutibacterium acnes is a gram-positive bacterium.
“bacterial cell wall associated with a mucopolysaccharide” means that the cell wall or a fragment, portion thereof is linked to a mucopolysaccharide. The linkage or bridge is formed in the process of production of the bacterial cell wall associated with a mucopolysaccharide as described herein. It is hypothesized that the peptidoglycan (murein) forming the bacterial cell wall, interacts or links with mucopolysaccharides.

As used herein, the terms “fragment of cell wall”, “fragment of wall” and “parietal fragment” of P. acnes are intended as synonymous.

Advantageously, the link or bridge between peptidoglycans and mucopolysaccharide forms a complex referred also as conjugate in the conditions of the herein disclosed process.
the term “association” as used herein means the bacterial cell wall or fragment/lysate of Cutibacterium acnes deposited with accession number DSM 28251 linked or associated with a mucolysaccharide.
the term “associated” and “linked” or “bound” as used herein are interchangeable and considered as synonyms and means the formation of a link, preferably with formations of bounds, between the bacterial wall and the mucopolysaccharide, especially hyaluronic acid. In certain passages the above terms are meant to designate cell wall fragment associated to the mucopolysaccharide according to the invention.
the terms “fragment” and “fraction” as used herein are synonymous when referred to the bacterial cell wall.
the term “lysate” referred to the bacterial cell wall means fragments of the cell wall obtained by disruption according to anyone of the herein disclosed disruption techniques.

Specifically, a bacterial cell wall fragment linked to a mucopolysaccharide as herein disclosed comprises a cell wall peptidoglycan of the C. acnes strain DSM 28251 linked to a mucopolysaccharide.

In certain embodiments, the cell wall fragment of C. acnes strain DSM 28251 comprises cell wall peptidoglycan and lipoteichoic acid and/or teichoic acid linked to a mucopolysaccharide.
“growth medium” (synonyms: medium, growth medium/cultivation broth/growth broth) refers to a substrate containing all the compounds (factors) required by a microorganism, especially a bacterium such as a gram+ bacterium for the cell replication resulting in the increasing of the single cells number and growth of the population. The factors required by the microorganisms for their growth in the medium belong mainly to the categories: carbon source, similar nitrogen source (constituted both by ammonia and free amino acids, the last also known as FAN), vitamins and salts (trace elements). Typical carbon sources are sugarcane molasses, beet molasses, barley malt extract and wheat malt extract.
“carrier” refers to an excipient, vehicle, diluent or adjuvant, which may or may be present in the composition of the invention.
“nutritional product” means a product, which improves the nutritional status and may be used to support or improve the functional activity of one or more organs or the functionality of the human body within the physiological boundaries.
“selected bacterial strain” or “strain” as mentioned herein means the strain of the invention deposited at the DSMZ with accession number DSM 28251.
the terms “bacterial cell wall”, “bacterial wall” “wall of the bacterial strain” “parietal wall”, as used herein, have the same meaning and relate to the cellular wall or a fragment or portion or lysate thereof of the bacterial strain Cutibacterium acnes deposited with accession number DSM 28251.

For the purposes of this invention, the bacterial cell wall herein disclosed may be entire or disrupted into parts or fragments or portions.

The term “fragment” or “lysate” means a portion of the bacterial cell wall of the DSM 28251 strain.

### PHARMACEUTICAL COMPOSITIONS

The herein described bacterial cell wall of strain DSM 28251 associated/linked to a mucopolysaccharide is advantageous for industrial application in the preparation of pharmaceutical compositions, medical devices, especially for topical application.

In accordance with an aspect, the present invention relates to a pharmaceutical composition comprising a bacterial cell wall of strain DSM 28251 linked/associated to a mucopolysaccharide as defined herein and a pharmaceutically or physiologically acceptable excipient.

The physiologically or pharmaceutically suitable carrier, diluent or excipient may be selected based on the route of administration for which the resulting pharmaceutical composition is intended.

The pharmaceutical compositions of the present invention encompass any compositions made by mixing a strain as herein defined, fragment thereof or postbiotic thereof according to the present invention and a pharmaceutically acceptable carrier. Such compositions are suitable for pharmaceutical use in an animal or human.

The pharmaceutical compositions, especially a medical device, of the present invention comprise a therapeutically effective amount of the bacterial cell wall of strain DSM 28251 associated/linked to a mucopolysaccharide and a pharmaceutically acceptable carrier.

A pharmaceutical composition may optionally contain other active ingredients. The term “carrier” refers to a vehicle, excipient, diluents, or adjuvant with which the therapeutic or active ingredient is administered. Any carrier and/or excipient suitable for the form of preparation desired for administration is contemplated for use with the strains/wall/postbiotic disclosed herein.

The carrier may take a wide variety of forms depending on the form of preparation desired for administration, e.g. oral or parenteral, including intravenous. In preparing the compositions for oral dosage form, any of the usual pharmaceutical media may be employed, such as, for example, water, glycols, oils, alcohols, flavouring agents, preservatives, colouring agents and the like in the case of oral liquid preparations, such as, for example, suspensions, elixirs and solutions; or carriers such as starches, sugars, microcrystalline cellulose, diluents, granulating agents, lubricants, binders, disintegrating agents, and the like in the case of oral solid preparations such as, for example, powders, hard and soft capsules and tablets, with the solid oral preparations being preferred over the liquid preparations.

In certain embodiments, the bacterial cell wall of strain DSM 28251 associated to a mucopolysaccharide of the present invention can be combined as an active ingredient in intimate admixture with a suitable pharmaceutical carrier and/or excipient according to conventional pharmaceutical compounding techniques.

The compositions include compositions or medical devices suitable for parenteral, including subcutaneous, intramuscular, and intravenous, pulmonary, nasal, rectal, topical or oral administration. Suitable route of administration in any given case will depend in part on the nature and severity of the conditions being treated and on the nature of the active ingredient. An exemplary route of administration is the oral route.

The compositions may be conveniently presented in unit dosage form and prepared by any of the methods well-known in the art of pharmacy. The preferred compositions include compositions suitable for oral, parenteral, topical, subcutaneous, or pulmonary, in the form of nasal or buccal inhalation, administration. The compositions may be prepared by any of the methods well-known in the art of pharmacy.

The pharmaceutical compositions may be in the form of tablets, pills, capsules, solutions, suspensions, emulsion, powders, suppository, and as sustained release formulations.

If desired, tablets may be coated by standard aqueous or non-aqueous techniques. In certain embodiments, such compositions and preparations can contain at least 0.1 percent of strain. The percentage of active bacterial cell wall of strain DSM 28251 associated to a mucopolysaccharide in these compositions may, of course, be varied and may conveniently be between about 0.1 percent to about 60 percent 0.5 to 20% of the weight of the unit. The amount of active bacterial cell wall of strain DSM 28251 associated to a mucopolysaccharide in such therapeutically useful compositions is such that therapeutically active dosage will be obtained. The bacterial cell wall of strain DSM 28251 associated to a mucopolysaccharide can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavouring agent such as cherry or orange flavour. To prevent breakdown during transit through the upper portion of the gastrointestinal tract, the composition be an enteric coated formulation.

In the frame of the invention, the topical uses are preferred. Accordingly, in certain preferred embodiments, the composition is for the topical application. In this application, the composition containing strain/wall/postbiotic as defined herein may be applied on the skin of human beings.

Compositions for topical administration include, but are not limited to, ointments, creams, lotions, solutions, pastes, gels, sticks, liposomes, nanoparticles, patches, bandages and wound dressings. In certain embodiments, the topical formulation comprises a penetration enhancer.

Compositions for pulmonary administration include, but are not limited to, dry powder compositions consisting of the powder of a strain/fragment/postbiotic, and the powder of a suitable carrier and/or lubricant. The compositions for pulmonary administration can be inhaled from any suitable dry powder inhaler device known to a person skilled in the art.

The composition for topical application may be in solid, semisolid or fluid form. Suitable formulations in solid form include creams, gels, ointments, pastes, unguents, creams, patches.

The composition for local application in fluid form, may be in the form of lotions, gels, suspensions, emulsions.

Typically, the composition for topical use may contain an amount of the above identified bacterial strain from 0.00001% to 10%, from 0.0001 to 3%, from 0,1 to 2% weight with respect to the total weight of the composition.

In the case of fluid or semi-fluid formulations form, the bacterial strain can be diluted in a carrier in physiologically acceptable liquid form such as water, alcohol, hydroalcoholic or glyceryl solution or mixed with other liquids suitable for local application.

By way of an example, the compositions of the invention in liquid form can be prepared by dissolving or dispersing the bacterial strain or a byproduct thereof in water and/or alcohol. The liquid composition can be buffered to reach a pH range conveniently selected from 5 to 7 to be compatible with the pH of the skin and then filtered and packaged in suitable containers such as bottles or vials.

In one embodiment, the formulation for the local application is in the form of a cream or emulsion containing the bacterial strain carried in a suitable excipient.

According to other embodiments, the composition of the invention is in form for systemic administration in particular for oral administration. In these cases, the composition contains the bacterial strain as previously defined and one or more vehicles or excipients suitable for systemic administration.

Administration of the compositions is performed under a protocol and at a dosage sufficient to reduce the target disease in the subject.

In some embodiments, in the pharmaceutical compositions or medical devices of the present invention the active principle or active principles are generally formulated in dosage units. The dosage unit may contain from 0.00001 to 1000 mg of bacterial cell wall of strain DSM 28251 associated to a mucopolysaccharide per dosage unit for daily administration.

In some embodiments, the amounts effective for topical formulation will depend on the severity of the disease, disorder or condition, previous therapy, the individual's health status and response to the drug. In some embodiments, the dose is in the range from 0.001% by weight to about 60% by weight of the formulation.

When used in combination with one or more other active ingredients, the bacterial cell wall of strain DSM 28251 associated to a mucopolysaccharide of the present invention and the other active ingredient may be used in lower doses than when each is used singly.

With respect to formulations with respect to any variety of routes of administration, methods and formulations for the administration of drugs are disclosed in Remington's Pharmaceutical Sciences, 17^{th} Edition, Gennaro et al. Eds., Mack Publishing Co., 1985, and Remington's Pharmaceutical Sciences, Gennaro A R ed. 20^{th} Edition, 2000, Williams & Wilkins PA, USA, and Remington: The Science and Practice of Pharmacy, 21^{st} Edition, Lippincott Williams & Wilkins Eds., 2005; and in Ansel's Pharmaceutical Dosage Forms and Drug Delivery Systems, 8^{th} Edition, Lippincott Williams & Wilkins Eds., 2005, which are herein incorporated as reference.

In certain embodiments, the composition of the invention for oral administration is a nutritional or dietetic or nutraceutical product.

Examples of embodiments and preferred procedures of the present invention are described below to illustrate the invention.

### EXAMPLE 1

Survival rate of Galleria mellonella larvae inoculated with supernatants from S. aureus BAA1680 and S. aureus ATCC 29213 cultures

### Premises

The pathogenicity of Staphylococcus aureus in atopic dermatitis (AD) and other inflammatory skin diseases is linked to its ability to produce many virulence factors, including secreted toxins, enzymes, and antigens associated with the cell surface. These factors allow this bacterium to evade the host's natural defences.

The cutaneous response to this insult involves a complex biological and molecular cascade of events, including inflammation, granulation tissue formation, re-epithelialization, and angiogenesis.

This study aims to verify that modified hyaluronic acid (bound to the bacterial wall of the C. acnes strain) is able to inactivate toxins/catabolites produced by S. aureus. For this purpose, Galleria mellonella larvae were used as a validated model for the study of bacterial toxins.

(Cutuli, M. A., Petronio Petronio, G., Vergalito, F., Magnifico, I., Pietrangelo, L., Venditti, N., & Di Marco, R. (2019). Galleria mellonella as a consolidated in vivo model hosts: new developments in antibacterial strategies and novel drug testing. Virulence, 10(1), 527-541.

Champion, O. L., Wagley, S., & Titball, R. W. (2016). Galleria mellonella as a model host for microbiological and toxin research. Virulence, 7(7), 840-845.)

### Materials and Methods

The strains of S. aureus BAA1680 and S. aureus ATCC 29213 were reactivated in Tryptic Soy Broth (TBS) and grown overnight at 37° C. until reaching a bacterial density of 109 CFU/ml determined using spectrophotometry (OD 600).

The broth culture was centrifuged at 4° C. for 20 minutes at 16,000 rpm. Subsequently, the supernatant was filtered (0.22 μm) to ensure the complete elimination of the bacterial cells.

The presence of the toxin was evaluated by a Bradford protein assay, using the bacteria-free TSB as a negative control.

The supernatant was first diluted in saline solution in a ratio of 1:2 and subsequently mixed with the bacterial cell wall of C. acnes DSM 28251 associated with hyaluronic acid (1:5 v/v). All suspensions obtained were incubated at 37° C. for 1 hour and 4 hours. After incubation, all suspensions were centrifuged at 4° C. for 20 minutes at 16,000 rpm to retrieve only the supernatants used in the subsequent in vitro and in vivo tests.

The larvae were selected based on weight and size and divided into 5 experimental groups (consisting of 20 specimens), each treated with the suspensions previously obtained. The inoculation was performed through injection, using a repeating dispenser equipped with insulin syringes (BD, Wellington) and an ultra-fine 1 ml needle through the posterior proleg of the larvae. After injection, the larvae for each condition were incubated in a petri dish at 35° C., and survival was observed over the following 96 hours.

### Results

The results obtained from monitoring the survival of larvae inoculated with the supernatant in contact with bacterial cell wall of C. acnes DSM 28251 linked with hyaluronic acid supernatant showed a higher survival rate than larvae inoculated with the untreated medium containing catabolites.

These results evidence that the bacterial cell wall of C. acnes DSM 28251 linked with hyaluronic acid interferes with the pathological mechanisms associated with the toxins produced by S. aureus.

### EXAMPLE 2

A delipidated wall fragment of C. acnes DSM 28251 was prepared in accordance with the following step:

1. Broth culture Strain C. acnes DSM 28251

2. Inactivation of the bacterial culture by heat (80° for 60 minutes)

3. Recovery of the pellet by centrifugation

4. Delipidization of the pellet, to remove potential fractions with immunogenic activity, using organic solvents

5. Disintegration of the cell pellet by homogenizer such as ultraturrax to obtain bacterial fragments

6. The suspension of cell fragments is subjected to centrifugation and only the supernatant is recovered

7. The supernatant is brought to a temperature of 80° C. for 1 minute

8. The resulting solution is added to a saturated 40% solution of ammonium sulfate

9. After precipitation at 4° C. over night the precipitate is collected by centrifugation

10. The bacterial fragment is purified by dialysis and subsequently lyophilized

1.-15. (canceled)

16. A bacterial cell wall or fragment thereof linked to a mucopolysaccharide wherein the bacterial strain is gram-positive and is Cutibacterium acnes deposited with accession number DSM 28251 at the International Deposit Authority Leibniz-Institut DSMZ.

17. The bacterial cell wall or fragment thereof linked to a mucopolysaccharide of claim 16 wherein the mucopolysaccharide is selected from heparin, heparan sulfate; chondroitin sulfate, dermatan sulfate; keratan sulfate; hyaluronic acid and chitosan and physiologically salts thereof.

18. The bacterial cell wall or fragment thereof linked to a mucopolysaccharide of claim 16 wherein the mucopolysaccharide is selected from hyaluronic acid, chondroitin-4-sulfate, chondroitin-6-sulfate, dermatan sulfate, chitosan, heparin sulfate, heparin, keratan-sulfate and physiologically acceptable salts thereof and preferably is hyaluronic acid or a salt thereof.

19. The bacterial cell wall or fragment thereof linked to a mucopolysaccharide according to claim 16 wherein the strain of Cutibacterium acnes DSM 28251 is obtained by mechanical or non-mechanical disruption of the cell wall.

20. The bacterial cell wall fragment linked with a mucopolysaccharide according to claim 16 wherein a cell wall peptidoglycan of the C. acnes strain DSM 28251 is linked to a mucopolysaccharide.

21. A medicament comprising the bacterial cell wall or fraction or lysate thereof associated with a mucopolysaccharide of claim 16.

22. A composition comprising an effective amount of a bacterial cell wall or fragment thereof linked with a mucopolysaccharide according to claim 16 and a physiologically acceptable carrier.

23. A composition according to claim 22 for topical use wherein the composition is in the form of a cream, foam, ointment, paste, powder, gel, solution, ovum, douche or emulsion.

24. A method for preventing or treating an immunological skin or mucosae disease in a subject comprising the topical application of a bacterial cell wall or fragment thereof linked with a mucopolysaccharide wherein the bacterial strain is gram-positive and is Cutibacterium acnes deposited with accession number DSM 28251 at the International Deposit Authority Leibniz-Institut DSMZ.

25. The method according to claim 24 wherein the immunological skin or mucosae disease is an allergic disease, an autoimmune disease, an inflammatory disease or an infectious disease of the skin.

26. The method according to claim 25 wherein the skin disease is eczema, atopic dermatitis, acne, seborrheic dermatitis, rosacea, psoriasis, erythema or cutaneous rash.

27. The method according to claim 25 wherein the infectious disease is a bacterial or fungal infection of the skin or mucosa, especially an infection of Candida.

28. The method according to claim 25 wherein the immunological mucosae disease is a gynecological disease.

29. The method according to claim 25 wherein the gynecological disease is vaginitis, vaginal infection, vaginal inflammation.

30. The method according to claim 29 wherein the vaginal infection is bacterial vaginosis, vulvar vaginal candidiasis, mixed vaginitis, vulvar vaginal infection.
